(19) United States
(12) Patent Application Publication
(10) Pub. No.: US 2009/0209941 A1
(43) Pub. Date: Aug. 20, 2009

(54) Implant deployment catheter

(75) Inventors: Steen Aggerholm, St. Heddinge (DK); Frank K. Christiansen, Haslev (DK)

Correspondence Address: COOK GROUP PATENT OFFICE, P.O. BOX 2269, BLOOMINGTON, IN, 47402, US

(73) Assignees: William Cook Europe, ApS, Bjaeverskov (DK); Cook Incorporated, Bloomington, IN (US)

(21) Appl. No.: 12/378,347

(22) Filed: Feb. 13, 2009

Related U.S. Application Data
(60) Provisional application No. 61/066,300, filed on Feb. 19, 2008.

(51) Int. Cl. A61M 25/00

(52) U.S. Cl. 604/523

(57) ABSTRACT

A rapid exchange implant deployment catheter includes a relatively stiff hypotube (12) and a relatively flexible distal tube (14). A side port (15) for a guide wire is provided towards the distal end of a transition zone (16) between the hypotube (12) and the distal tube (14). The distal end of the hypotube (12) is laser cut to form a plurality of slits (18) between which are formed a plurality of projections (11, 11′). At least one of the projections (11′) extends into the distal tube 14 to a point beyond the position of the side port (15).

### CROSS-REFERENCE TO RELATED APPLICATIONS

This application claims priority of provisional application Ser. No. 61/066,300, filed Feb. 19, 2008.

### TECHNICAL FIELD

The present invention relates to an implant deployment catheter. In particular it relates to a catheter that may be used in a rapid exchange system.

### BACKGROUND OF THE INVENTION

Catheters have found widespread use in medical procedures, such as percutaneous transluminal coronary angioplasty (PTCA) or for delivery of an implant such as a stent, a stent-graft or an occlusion device. Most catheters are guided to the application site by sliding the catheter along a guide wire, which has been carefully advanced and arranged within the patient. During advancement of the catheter along the guide wire, it is important to keep the guide wire steady. Ordinary catheters are guided to the application site in a patient by sliding the catheter along a guide wire extending all the way through a lumen of the catheter from the proximal end to the distal end thereof. To enable the physician to hold or manipulate the guide wire during advancement of the catheter along the guide wire, it is necessary to have an excess length of guide wire. The guide wire must hence have a length of about twice the length of the catheter, e.g. 3 m in total, which greatly complicates the procedure. An important sub-category of catheters are catheters of the so-called rapid exchange type, which greatly facilitate operation, especially exchange of catheters if it is found during a procedure that a different kind or size of catheter is needed for the specific purpose. In the rapid exchange catheter, the guide wire only passes through a minor part of the catheter at the distal end thereof, whereas along a majority of the catheter, the guide wire runs in parallel with the catheter. Hence it is not necessary to have an excess length of guide wire. However the rapid exchange catheter provides some challenges, especially with regard to resistance to kinking of the catheter.

An important feature of catheters is the transmission of force, the so-called push force, from the proximal end to the distal end of the catheter. This transmission significantly affects the physician's ability to direct the distal end of the catheter into a body lumen of a patient by manipulating the proximal end thereof. Another important feature of catheters is the flexibility of the distal end to bend and conform to the body lumen wall without causing any injury to the lumen wall. Hence catheters, especially of the rapid exchange type, are commonly manufactured of a metal proximal shaft portion of relatively high stiffness and known as a “hypotube”, and a relatively flexible plastics distal portion bonded to the hypotube. An abrupt change of properties between the hypotube and the distal portion, however, increases the risk of twist and kinking. Hence it is desirable to provide a good and simple transition between the relatively stiff hypotube to the relatively more flexible distal section to provide a sufficient resistance to twist and kinking while maintaining flexibility and ability to bend.

Known methods of providing a transition between a stiff proximal portion and a more flexible distal portion are disclosed in U.S. Pat. No. 5,743,876, US 2007/0125709, U.S. Pat. No. 6,575,958, EP 1 084 728, U.S. Pat. No. 5,938,653 and U.S. Pat. No. 6,685,720.

In one prior art method of providing a transition between a hypotube and a distal tube, a tapered piece of metal is attached to the distal end of the hypotube and to the proximal end of the distal tubing leading to the dilator tip. However, this increases the complexity of the device. Furthermore, this arrangement does not provide effective transition in flexibilities of the device in all radial directions in which it is typically deflected during the deployment procedure.

### SUMMARY OF THE INVENTION

The present invention seeks to provide an improved implant deployment catheter.

According to a first aspect of the present invention, there is provided an implant deployment catheter including a first longitudinal portion and a second longitudinal portion, the first longitudinal portion being less flexible than the second longitudinal portion; and a transition zone between the longitudinal portions; wherein the transition zone includes a plurality of projections extending from the first longitudinal portion into the second longitudinal portion, and wherein at least one of the projections extends into the second longitudinal portion to an extent greater than the other projections.

Having at least one of the projections extending into the second longitudinal portion to an extent greater than the other projections allows additional resistance to kinking to be provided where desired. This may be particularly useful where the catheter is a rapid exchange implant deployment catheter.

According to a second aspect of the present invention, there is provided a rapid exchange implant deployment catheter including a first longitudinal portion and a second longitudinal portion, the first longitudinal portion being less flexible than the second longitudinal portion; and a transition zone between the longitudinal portions, wherein the transition zone includes a plurality of projections extending from the first longitudinal portion into the second longitudinal portion.

Kinking is particularly problematic where an implant deployment catheter is of the rapid exchange type. Providing a plurality of projections helps to provide a smooth transition between two longitudinal portions of differing flexibility.

Preferably at least one of the projections extends into the second longitudinal portion to an extent greater than the other projections.

The transition zone may include a side port for a guide wire, and at least one of the projections may extend beyond the side port. As the location of the side port provides a particular point of weakness in a catheter, extending at least one of the projections beyond this provides additional resistance to kinking at a particularly vulnerable point.

In an embodiment, the first longitudinal portion is a hypotube and the second longitudinal portion is a distal tube.

In a particularly preferred embodiment, the catheter is a rapid exchange implant deployment catheter including a hypotube and a distal tube, the hypotube being less flexible than the distal tube; and a transition zone between the hypotube and the distal tube, wherein the transition zone includes a plurality of projections extending from the hypotube into the distal tube, and wherein at least one of the projections extends beyond a side port in the transition zone.

According to a third aspect of the present invention, there is provided a rapid exchange implant deployment catheter including a hypotube and a distal tube, the hypotube being less flexible than the distal tube; and a transition zone between the hypotube and the distal tube, wherein the transition zone includes a plurality of projections extending from the hypotube into the distal tube, and wherein at least one of the projections extends beyond a side port in the transition zone.

According to a fourth aspect of the present invention, there is provided an implant deployment assembly including a catheter as described above.

### BRIEF DESCRIPTION OF THE DRAWINGS

An embodiment of the present invention is described below, by way of example only, with reference to the accompanying drawings, in which:

FIG. 1 is a side view of a rapid exchange catheter in accordance with an embodiment of the invention; and

FIG. 2 shows an enlargement of the transition zone of the catheter of FIG. 1.

### DETAILED DESCRIPTION

It is to be understood that the Figures are schematic and do not show the various components in their actual scale. In many instances, the Figures show scaled up components to assist in the understanding of the features disclosed therein.

In this description, when referring to a deployment assembly, the term distal is used to refer to an end of a component which in use is furthest from the surgeon during the medical procedure, including within a patient. The term proximal is used to refer to an end of a component closest to the surgeon and in practice in or adjacent an external manipulation part of the deployment or treatment apparatus.

On the other hand, when referring to an implant such as a stent or stent graft, the term proximal refers to a location that in use is closest to the patient's heart, in the case of a vascular implant, and the term distal refers to a location furthest from the patient's heart.

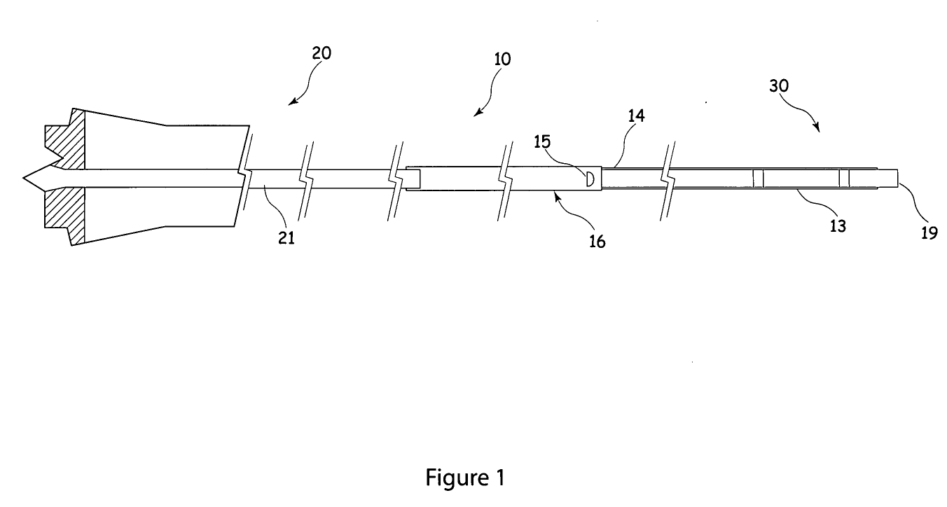

FIG. 1 shows a rapid exchange catheter according to an embodiment of the invention. The total length of the catheter is, in some cases, about 1.4 m. The catheter 10 has a proximal end 20 and a distal end 30. The catheter 10 comprises a tubular metal shaft body 12, also known as a hypotube, made of a Nitinol alloy. An inflation lumen 21 extends through the full length of the metal shaft body 12.

A plastics distal end portion 14 is attached to the distal end of the tubular metal shaft body 12 by bonding. The plastics distal end portion 14 is thus axially aligned with the tubular metal shaft body 12. The plastics distal end portion 14 comprises a side port 15 for a guide wire (not shown) at a proximal side of an inflatable balloon 13, so the guide wire may extend through the most distal part of the catheter to the distal end opening 19. The region where the hypotube 12 is joined to the distal tube 14 is known as the transition zone 16.

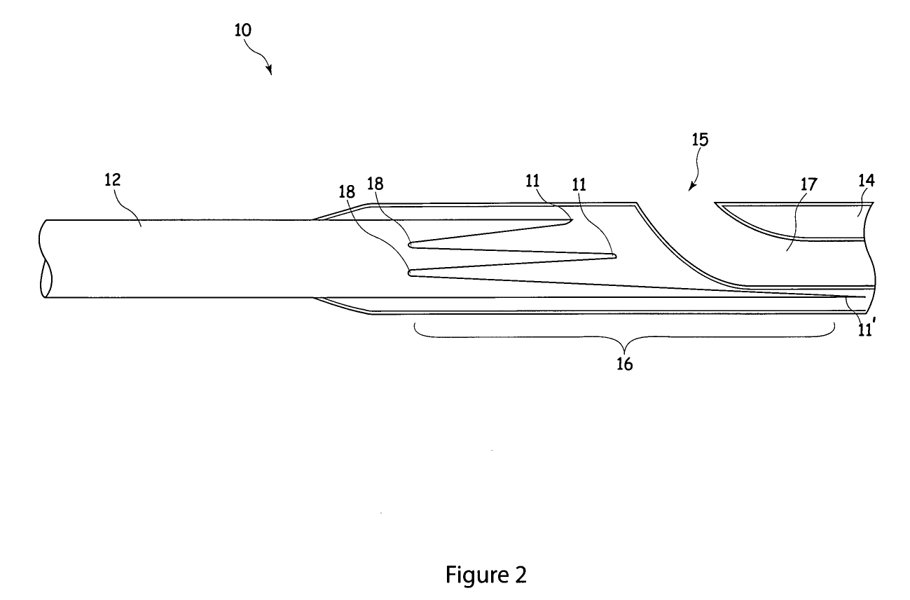

FIG. 2 shows the transition zone 16 in more detail and rotated 90° about the longitudinal axis of the catheter compared to FIG. 1.

Towards the distal end of the transition zone, there is formed in the distal tube 14, a side port 15 for a guide wire. The side port 15 communicates with a longitudinally extending lumen 17 for a guide wire.

The distal end of the hypotube 12 is laser cut to form four longitudinally and proximally extending slits 18, which define four distally extending projections 11, 11′. These are referred to hereinafter as “top” and “bottom”, with two “side” projections (of which only one can be seen in FIG. 2). The distal end of the hypotube 12 thus resembles a circumferential fork with distally extending teeth 11, 11′.

The hypotube 12 has an outside diameter that corresponds to the inner diameter of the distal tube 14. The connection is secured by means of gluing or welding. The projections 11, 11′ are thus arranged around the internal surface of the distal tube 14.

The projections 11, 11′ have different lengths to provide different flexibilities around the transition zone 16 in different radial directions. At least one of the projections 11′ located circumferentially opposite the side port 15 (the bottom projection), extends distally to a greater extent than the other projections 11. The bottom projection 11′ thus extends further into the distal tube 14, and to a point approximately 3 cm distally beyond the side port 15 for the guide wire. The projections 11 may typically be up to around 7 to 9 cm long, with the bottom projection 11′ typically being up to approximately 12 cm in length.

In use, the stiffer hypotube 12 provides the necessary pushability to enable the distal tube 14 to reach the site at which an implant is to be deployed. The distal tube 14 is flexible enough to negotiate the patient's vasculature. At the transition zone 16, the projections 11, 11′ that extend into the proximal end of the distal tube 14, reduce the possibility of kinking between the hypotube 12 and the distal tube 14 at the transition zone 16.

There are several advantages to the above described embodiment. The projections 11, 11′ provide a smooth transition between the relatively stiff hypotube 12 and the relatively flexible distal tube 14. This lowers the risk of the catheter kinking at the transaction zone 16. The projection 11′ that extends to a greater extent in the distal direction across a side port 15 for a guide wire provides additional reinforcement.

This arrangement does not require any gluing or welding on the inside surface of the hypotube 12. Furthermore, no additional components are required.

Of course, many modifications may be made to the above described embodiment. In particular, the number and precise length of the projections 11, 11′ may be altered to suit a particular application. Transition properties can thus be tailor-made to suit a particular specific purpose. For example, the middle projections 11 may have a length intermediate that of the top projection 11 and the bottom projection 11′. The distal tips of the projections 11, 11′ may thus follow the general shape of the side port 15/guide wire lumen 17. The middle projections 11 could be omitted altogether, and the hypotube 12 may be provided only with top and bottom projections.

In another modification the projections 11, 11′ of the hypotube 12 may be embedded in the wall of the distal tube 14. However, this is less preferred as the flexibility would be lower.

The disclosures in U.S. 61/066,300, from which this application claims priority, and in the abstract accompanying this application are incorporated herein by reference.

What is claimed is:

1. An implant deployment catheter including a first longitudinal portion and a second longitudinal portion, said first longitudinal portion being less flexible than said second longitudinal portion; and a transition zone between said longitudinal portions; wherein said transition zone includes a plurality of projections extending from said first longitudinal portion into said second longitudinal portion, and wherein at least one of said projections extends into said second longitudinal portion to an extent greater than other projections.

2. A catheter as claimed in claim 1, wherein said catheter is a rapid exchange implant deployment catheter.

3. A catheter as claimed in claim 1, wherein said transition zone includes a side port for a guide wire, and wherein at least one of said projections extends beyond the side port.

4. A catheter as claimed in claim 1, wherein said first longitudinal portion is a hypotube and wherein said second longitudinal portion is a distal tube.

5. A rapid exchange implant deployment catheter including a first longitudinal portion and a second longitudinal portion, said first longitudinal portion being less flexible than said second longitudinal portion; and a transition zone between said longitudinal portions, wherein said transition zone includes a plurality of projections extending from said first longitudinal portion into said second longitudinal portion.

6. A catheter as claimed in claim 5, wherein at least one of said projections extends into said second longitudinal portion to an extent greater than other projections.

7. A catheter as claimed in claim 5 wherein said transition zone includes a side port for a guide wire, and wherein at least one of said projections extends beyond the side port.

8. A catheter as claimed in claim 5 wherein said first longitudinal portion is a hypotube and wherein said second longitudinal portion is a distal tube.

9. A rapid exchange implant deployment catheter including a hypotube and a distal tube, said hypotube being less flexible than said distal tube; and a transition zone between said hypotube and said distal tube, wherein said transition zone includes a plurality of projections extending from said hypotube into said distal tube, and wherein at least one of said projections extends beyond a side port in said transition zone.

10. An implant deployment assembly including a catheter as claimed in claim 1.

11. An implant deployment assembly including a catheter as claimed in claim 5.

12. An implant deployment assembly including a catheter as claimed in claim 9.
